(19) United States
(12) Patent Application Publication
(10) Pub. No.: US 2008/0269555 A1
(43) Pub. Date: Oct. 30, 2008

(54) Intragastric Balloon With Extraction Reinforcement

(75) Inventors: Pascal Paganon, Serezin du Rhone (FR); Jean-Paul Gilbert Ricol, Saint Germain sur L'Arbresle (FR)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: Compagnie Europeenne d'Etude et de Recherche de Di spositifs pour l'lmplantation par Laparoscopie, Vienne (FR)

(21) Appl. No.: 11/817,071

(22) PCT Filed: Feb. 24, 2005
(86) PCT No.: PCT/FR2005/000439

§ 371 Date: May 14, 2008

(51) Int. Cl. A61M 29/02; A61B 1/018

(52) U.S. Cl. 600/104; 606/191; 606/192

(57) ABSTRACT

An intragastric balloon (1) for treating obesity, designed to be implanted in a patient's stomach to reduce the stomach volume, the balloon (1) being delimited by a surface envelope (3) and wherein at least one fraction (6, 7) of the surface envelop (3) is associated with a reinforcement (8), to form a reinforced portion designed to extract the balloon (1) out of the stomach by gripping the reinforced portion with an endoscopic extracting tool (2).

### PRIORITY CLAIM

This patent application is a U.S. National Phase of International Patent Application No. PCT/FR2005/000439, filed Feb. 24, 2005, the disclosure of which is incorporated herein by reference in its entirety.

### FIELD

The present disclosure relates to artificial devices for treating obesity, in particular, morbid obesity, and most particularly to devices that artificially reduce the volume of the gastric cavity in order to give the patient a sensation of being sated rapidly.

The present disclosure relates more particularly to an intragastric balloon for treating obesity, for implanting in the stomach of a patient in order to reduce the volume of the stomach, the balloon being defined by a surface envelope.

The present disclosure also provides a kit comprising firstly an intragastric balloon for treating obesity, that is for implanting in the stomach of a patient in order to reduce the volume of the stomach, the balloon being defined by a surface envelope, and secondly an endoscopic extractor tool.

### BACKGROUND

For the purpose of treating patients suffering from obesity, and in particular those presenting a weight/size ratio that does not make it necessary to use surgical devices and methods that are invasive, cumbersome, and traumatic, such as surgically implanting a gastric ring, or likewise for treating patients in which their excess weight is so great that it constitutes a danger to undertaking surgery, it is known to implant a foreign body directly in the stomach of the patient, the foreign body being of a volume that is sufficient to reduce the space available for food, and also reducing the transit speed of food.

Such foreign bodies can be implanted endoscopically via the mouth, and they are generally in the form of balloons known as intragastric balloons. Such balloons are generally in the form of a flexible pouch made of a biocompatible elastomer material, and they are implanted directly in the stomach of a patient.

Once the balloon has been implanted inside the stomach, the flexible pouch, forming the surface envelope of the balloon, can be filled with an inflation fluid, such as physiological liquid, to give the balloon its functional shape, i.e., to give the balloon a utilization shape and volume that enable it to occupy a large fraction of the space that would otherwise be available for food.

Such intragastric balloons are widely known, and although they provide results that are of interest in terms of weight loss, since they reduce the rate at which food passes through and they contribute effectively to giving rise quickly to a sensation of being sated, they nevertheless suffer from drawbacks that are not negligible.

In particular, withdrawing the balloon from the stomach, which is necessary after it has been implanted for a few weeks or a few months, turns out to be lengthy and difficult to perform for the practitioner.

The balloon is withdrawn via the natural passages (mouth and esophagus) using an endoscopic clamp that is lowered via the operator channel of an endoscope from outside the patient's body to the balloon situated inside the patient's stomach. Since the operator channel of the endoscope is of very small section (e.g., having a diameter of less than 2.8 millimeters), the endoscopic clamp for gripping the balloon in order to withdraw the balloon from the stomach is itself necessarily of very small dimensions. That means that the jaws or hooks of the clamp are particularly sharp, pointed, and hard.

When using conventional endoscopic clamps commonly used by practitioners, it is very difficult to ensure that the clamp grips the balloon firmly so as to be able to pull the balloon out from the stomach by pulling the clamp out from the patient's body. The surface envelope of the balloon is made of a material that is flexible and non-traumatic, generally of silicone. When that material is subjected to the gripping and traction action of the clamp, the material tears, given the sharp nature of the clamp due to its small size. When the silicone tears, grip is lost and the practitioner needs to make numerous repeat attempts before being able to bring the balloon out from the stomach, thereby lengthening the duration of the operation, which is harmful for the patient.

The above-described difficulties of gripping the balloon by means of an endoscopic clamp are made worse by the fact that the material constituting the surface envelope of the balloon is rendered slippery by spending time in the stomach, thus making it even more difficult to get a firm grip between the clamp and the balloon in order to be able to remove the balloon.

The small dimensions of the endoscopic clamp also mean that it can exert only a small force on a zone of small surface area. This impossibility of using known endoscopic clamps to exert broad and firm grip on the balloon requires the practitioner to proceed with numerous attempts before being able to extract the balloon from the stomach endoscopically.

### SUMMARY

The present disclosure provides an intragastric balloon that can be extracted quickly and easily from the stomach of the patient in conventional manner by endoscopic means.

The present disclosure describes several exemplary embodiments of the present invention.

One aspect of the present disclosure provides an intragastric balloon for implanting in the stomach of a patient in order to reduce the volume of the stomach and thereby treat obesity, the balloon comprising a surface envelope wherein at least a fraction of the surface envelope is associated with a reinforcement for forming a reinforced fraction dedicated to extracting the balloon from the stomach by gripping the reinforced fraction with the help of an endoscopic extractor tool.

Another aspect of the present disclosure provides a kit, comprising a) an intragastric balloon for implanting in the stomach of a patient in order to reduce the volume of the stomach thereby treating obesity, the balloon comprising a surface envelope, and b) an endoscopic extractor tool, wherein a fraction of the surface envelope is associated with a reinforcement, for forming a reinforced fraction that is dedicated to extracting the balloon from the stomach by gripping the reinforced fraction with the help of the endoscopic extractor tool.

A further aspect of the provides an intragastric balloon having an outer envelope that presents a regular shape and that lends itself particularly well to folding operations in order to implant the balloon endoscopically.

An additional aspect of the present disclosure provides an intragastric balloon of construction that is particularly compact.

Yet another aspect of the present disclosure provides an intragastric balloon that is non-traumatic.

Another aspect of the present disclosure provides an intragastric balloon that is particularly simple to fabricate.

A further aspect of the present disclosure provides an intragastric balloon that is of construction that is particularly compact and strong.

An additional aspect of the present disclosure provides an intragastric balloon that is made using elements that are standard and inexpensive.

Yet another aspect of the present disclosure provides an intragastric balloon that is suitable for being gripped easily and securely by conventional endoscopic extractor tools.

Another aspect of the present disclosure provides an intragastric balloon that, while being particularly flexible and lightweight, can easily be gripped securely with the help of a conventional endoscopic extractor tool.

A further aspect of the present disclosure provides an intragastric balloon that, while being of sufficient volume, is particularly light in weight and well supported by the patient.

An additional aspect of the present disclosure provides a kit comprising an intragastric balloon and an endoscopic extractor tool, the kit making it easy to extract the balloon quickly from the patient's stomach.

Yet another aspect of the present disclosure provides a kit comprising an intragastric balloon and an endoscopic extractor tool that enable the practitioner to perform balloon extraction from the patient's stomach using a procedure that is conventional and well-proved.

### BRIEF DESCRIPTION OF THE DRAWING

Other aspects of the present disclosure appear on reading the following description, and with the help of the accompanying drawing given purely by way of non-limiting illustration.

FIG. 1 is a sectional view showing an exemplary embodiment of an intragastric balloon in the process of being gripped by an endoscopic clamp.

### DETAILED DESCRIPTION

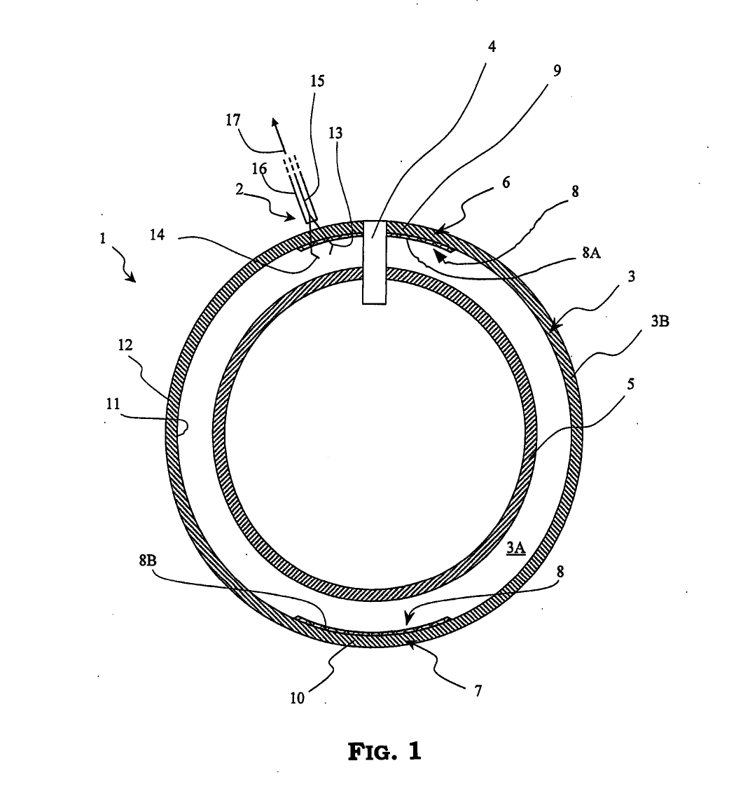

FIG. 1 shows an intragastric balloon 1, together with certain details concerning its implementation, the balloon 1 being in the process of being subjected to the gripping action of an endoscopic extractor tool 2, constituted in this example by an endoscopic clamp.

The intragastric balloon 1 is designed for treating obesity, and for this purpose it is designed to be implanted in the stomach of a patient in order to reduce the volume of the stomach, insofar as the balloon occupies a major fraction of the space available for food.

In the preferred exemplary embodiment shown in FIG. 1, the intragastric balloon 1 presents an expandable nature, i.e., the balloon is made using materials that are sufficiently flexible, e.g., materials based on elastomers and, in particular, on silicone, to enable the balloon to occupy both a folded or slack configuration (not shown) in which it occupies a small volume making it easier to implant, and also an expanded configuration in which it is expanded to a predetermined volume, e.g., of about 600 millimeters (mL) corresponding to its functional volume in use, as shown in FIG. 1.

As a general rule, an intragastric balloon 1 in accordance with the present disclosure is implanted in a conventional manner, as is well known to the person skilled in the art, by being passed endoscopically via the oral passages and the esophagus while it is in its folded or slack shape, with the balloon being expanded and put into place after the end of the endoscopic operation, once the balloon 1 is properly positioned in the patient's stomach.

In general, explanation of the balloon 1, i.e., removing the balloon from the patient's stomach after a stage of treatment, which might last for six months, for example, is performed endoscopically via the esophagus and the mouth. The operation of extracting the balloon 1 in accordance with the present disclosure might conventionally comprise a first stage of deflating the balloon, in which the balloon 1 is caused to pass from its expanded configuration to its folded or slack configuration, with this first stage being followed by an extraction operation proper, in which the balloon is caught by means of an endoscopic tool, and then the balloon is pulled out from the patient's stomach via the natural passages.

The intragastric balloon 1 in accordance with the present disclosure is defined by a surface envelope 3, which preferably forms a first flexible pouch defining a predetermined inside volume 3A, as shown in FIG. 1.

The surface envelope 3 is made from a material that is flexible and non-traumatic. Preferably, this material is an elastomer material, and, in particular, the material is based on biomedical grade silicone. In the context of the present disclosure, it is also possible to envisage that the surface envelope 3, e.g., made substantially entirely out of silicone, is covered over all or part of its surface in one or more protective films, for example films based on PARYLENE®

Once the balloon 1 is in its expanded configuration, the surface envelope 3 is substantially ellipsoidal in shape, and more preferably is substantially spherical in shape (as shown in FIG. 1). Naturally, other geometrical shapes could be envisaged without going beyond the scope of the present disclosure.

The intragastric balloon 1 includes a valve 4 associated with the surface envelope 3, i.e., preferably mounted on or fastened to the surface envelope 3. The valve 4 is for connecting to a source of fluid (not shown) for expanding the balloon 1 in the stomach by filling the balloon with the fluid.

As shown in FIG. 1, the intragastric balloon comprises at least first and second flexible pouches 3B, 5, the surface envelope 3 forming the first flexible pouch 3B as described above, while the second flexible pouch 5 is located inside the first pouch 3B, in its inside volume 3A, as shown in FIG. 1.

The second flexible pouch 5 forms means for shaping the first pouch 3B that is formed by the surface envelope 3. More precisely, the second pouch 5 is connected to the valve 4 so that introducing fluid, e.g., air, via the valve 4 serves to inflate the second pouch 5. The inflation of this second pouch 5 acts, in turn, like an “inner tube”, to cause the first pouch 3B formed by the surface envelope 3 to be inflated and put into shape.

Naturally, the present disclosure is not limited to this particular exemplary embodiment, but also applies to intragastric balloons having a single pouch suitable for being filled directly with fluid.

Nevertheless, implementing a two-pouch construction of the kind described above and shown in FIG. 1 is preferred since that enables the balloon to be filled with a fluid that is very light, such as air, while ensuring that the balloon has excellent leaktightness because of the presence of two pouches instead of one. Using two distinct pouches also makes it possible to deal separately with the function of leaktightness that relies mainly on the inner pouch 5, and the function of being non-traumatic and strong, that relies on the outer pouch 3B. For this purpose, the outer pouch 3B may be made, as described above, out of biomedical grade silicone, while the inner pouch 5 may be made of a material that presents gas-barrier properties, such as thermoplastic elastomer polyurethane.

According to one feature of the present disclosure, at least a fraction 6, 7 of the surface envelope 3 is associated with reinforcement 8 for forming a reinforced fraction dedicated to extracting the balloon 1 from the stomach by the reinforced fraction being gripped using an endoscopic extractor tool 2.

In other words, at least a portion of the surface envelope 3 is provided with reinforcement 8 such that the portion as reinforced in this way can facilitate extraction of the balloon 1 from the stomach when the extraction is performed by gripping the reinforced portion using an endoscopic extractor tool, and, in particular, a conventional endoscopic tool of the clamp type.

The surface envelope 3 is thus associated, at least locally, with reinforcement 8, the zone 6, 7 of the envelope as reinforced in this way presenting sufficient mechanical strength to enable the balloon 1 to be gripped and pulled with the help of an endoscopic gripper tool. The present disclosure naturally applies to circumstances in which only a single fraction of the envelope 3 is associated with reinforcement 8 (as shown in FIG. 1), and also to circumstances (not shown) in which the entire surface envelope 3 is associated with reinforcement, such that the entire surface envelope 3 is reinforced by the reinforcement.

The general principle of the present disclosure amounts to improving the strength of the zone of the balloon 1 that is to be subjected to the action of the endoscopic extractor tool 2, and, in particular, to improving the tear resistance of the zone to enable the tool to grasp the balloon firmly, preferably in a single attempt, for the purpose of pulling it out, without it being possible for the balloon 1 to escape from the extractor tool 2 under the effect of its own weight and/or of the stresses associated with the balloon being pulled out from the stomach.

When only a fraction 6, 7 of the envelope 3 is reinforced, as shown in FIG. 1, the fraction may be continuous, i.e., formed by a unitary portion of the envelope 3, or, on the contrary, the fraction may be distinct, i.e., formed by a plurality of distinct and separate portions of the envelope 3, and, for example, by two distinct portions, as shown in FIG. 1. When the fraction is discrete, the reinforcement 8 can also be discrete in nature, and can be made up of distinct and separate reinforcing elements corresponding respectively to the distinct portions of the envelope forming the fraction (as shown in FIG. 1).

Only a fraction 6, 7 of the surface envelope 3 is associated with reinforcement 8, and not the entire envelope 3, with the fraction 6, 7 comprising a first portion 9 of the envelope situated in the vicinity of the valve 4. The reinforcement 8 constitutes a first reinforcing element 8A associated with the first portion 9.

The valve 4 is easily identified visually using an endoscope, since the valve 4 forms an irregularity in the outside surface of the surface envelope 3. Starting from the position of the valve 4, the practitioner setting out to extract the balloon 1 from the stomach can easily deduce the position of the first reinforcing element 8A contributing to forming the reinforcement 8, and thus knows where to apply the endoscopic extractor instrument 2.

In addition to the above-mentioned first portion 9 of the envelope 3, the fraction 6, 7 of the surface envelope 3 that is associated with the reinforcement 8 also comprises a second portion 10 of the envelope 3 that is situated diametrically opposite from the first portion 9, as shown in FIG. 1. Under such circumstances, the reinforcement 8 comprises a second reinforcing element 8B distinct from the first reinforcing element 8A, but preferably of similar construction. The second reinforcing element 8B is associated with the second portion 10.

Thus, in the particular exemplary embodiment shown in FIG. 1, only two poles of the spherical pouch 3B formed by the surface envelope 3 are associated functionally with the reinforcement 8.

As shown in FIG. 1, the shape of the reinforcement 8 substantially matches the shape of the fraction 6, 7 of the surface envelope 3 with which the reinforcement 8 is associated.

In other words, the reinforcement 8, which is preferably in the form of a flexible membrane, extends along the fraction 6, 7 of the surface envelope 3 with which it is associated so that it does not project from the surface envelope 3, and therefore does not form any projection that might interfere with the regular and non-traumatic shape of the balloon 1.

This aspect contributes to the balloon 1 being well tolerated by the patient.

This aspect also makes it easier to fold the balloon 1 for endoscopic implantation. Because the reinforcement 8 is preferably permanently complementary in shape to the fraction 6, 7 of the surface envelope 3 with which it is associated, it does not constitute any impediment for folding the balloon 1 as tightly as possible, and thus does not prevent the balloon 1 from passing along the small-diameter passage constituted by the esophagus.

The reinforcement 8 is superposed on the fraction 6, 7 of the surface envelope 3 with which it is associated. More precisely, the reinforcement 8 is pressed against the surface envelope 3 so as to cooperate therewith to form, at least locally, a two-layer lamination, as shown in FIG. 1.

The surface envelope 3, and thus the fraction 6, 7 of the surface envelope 3 with which the reinforcement 8 is associated, presents an inside face 11 situated facing the inside of the balloon 1 and an opposite outside face 12. Preferably, the reinforcement 8 is superposed on the inside face 11.

This technical measure serves to confer an excellent non-traumatic nature on the balloon 1 in accordance with the present disclosure, since only the surface envelope 3, which is preferably made of a non-traumatic material such as silicone, is likely to come into contact with the stomach wall of the patient. Because the reinforcement 8 is contained inside the balloon 1, inside the internal volume 3A, the reinforcement 8 never comes into contact with biological tissues of the patient, thereby limiting any risk of traumatizing such tissues, where such risk stems essentially from the fact that the main function of the reinforcement 8 is to provide strength, which is not necessarily compatible with being non-traumatic. In other words, the positioning of the reinforcement 8 on the inside face 11 of the surface envelope 3 serves to protect the patient against any risk of trauma associated with the presence of the reinforcement 8.

The reinforcement 8 and the surface envelope 3 are associated with each other by mechanically connecting the reinforcement 8 to the surface envelope 3. This mechanical connection may be obtained by any means known to the person skilled in the art.

For example, the reinforcement 8 is preferably bonded to the fraction 6, 7 of the surface envelope 3 with which it is associated by means of adhesive. Preferably, the reinforcement 8 can be adhesively bonded over its entire area to the fraction 6, 7 of the surface envelope 3 with which it is associated. This connection between the reinforcement 8 and the surface envelope 3 over their entire contact interface serves to link the mechanical behaviors of the surface envelope 3 and of the reinforcement 8 together closely (particularly when folding), which can be advantageous for folding the balloon 1 as tightly as possible in order to implant it endoscopically.

Thus, in preferable manner and as shown in FIG. 1, the reinforcement 8 lines the inside face 11 of the surface envelope 3, at least locally.

Bonding means other than adhesive could naturally be envisaged, such as heat-sealing or stitching, for example, without thereby going beyond the scope of the present disclosure.

The present disclosure is not limited to reinforcement 8 being applied to the inside face 11 (or the outside face 12) of the surface envelope 3. It is entirely possible, without going beyond the scope of the present disclosure, for the reinforcement 8 to be disposed in the thickness of the fraction 6, 7 of the surface envelope 3 with which it is associated. Under such circumstances, the reinforcement 8 is embedded within a matrix formed by the material constituting the surface envelope 3. By way of example, such a configuration can be obtained by overmolding the reinforcement 8.

The reinforcement 8 comprises at least one textile piece.

In the exemplary embodiment shown in FIG. 1, the reinforcement 8 has two distinct textile pieces corresponding respectively to the first and second reinforcing elements 8A, 8B. Each of these textile pieces is substantially in the form of a spherical cap, for example, and is of curvature that matches that of the pouch 3B formed by the surface envelope 3.

The use of a textile material, and, in particular, a woven fabric, is particularly advantageous because it enables the balloon 1 to retain a flexible and lightweight nature, while reinforcing the balloon sufficiently in the zones that are to be gripped by the extractor tool 2.

Preferably, the textile piece used as reinforcement 8 presents a mesh size adapted to encouraging retention of the endoscopic extractor tool 2 by the textile piece. More particularly, the mesh in the textile piece should be sufficiently small to enable it to catch the endoscopic extractor tool 2. When the endoscopic tool 2 is made by a clamp built up of metal wires (cf. FIG. 1), the arrangement of the textile piece enables loops to form in which the jaws 13, 14 of the clamp 2 become hooked. This hooking provides additional safety avoiding the balloon 1 becoming separated from the clamp 2 while the balloon 1 is being pulled out from the stomach by the clamp 2.

As examples, the textile piece may comprise a polyester tulle or a woven (or non-woven) fabric made from polyamide fibers and/or aramid fibers. It is also possible to envisage using a “honeycomb” type fiber structure as a reinforcing part, in as well known in the art.

Naturally, other types of yarn and fabric could be used in the context of the present disclosure. As an alternative, the textile piece may comprise sheets or layers that are, among of things, woven, non-woven, knitted, or braided, the sheets or layers being made from yarn of any chemical nature, for example, based on polypropylene or on NYLON®.

In general, in order to implement the present disclosure, a textile should be selected that presents the best possible compromise between strength, which must be as high as possible, and fiber size, which must be as small as possible in order to encourage a firm grip of the textile by an endoscopic gripper tool.

Naturally, the use of a textile piece is purely optional and, as an alternative, the reinforcement 8 could comprise a film of plastics material presenting good resistance to tearing, or, in any event, resistance to tearing greater than that of the envelope 3, such as a film of thermoplastic elastomer polyurethane, for example.

In general, the reinforcement 8 should be selected, in particular, in application of the following criteria:
the reinforcement presents sufficient strength to enable the balloon 1 to be pulled out from the stomach by an endoscopic tool 2;
the reinforcement is sufficiently thin to enable the balloon to be folded tightly for implantation purposes; and
the reinforcement is sufficiently flexible to facilitate implanting the balloon via the natural tracts of the mouth and the esophagus.

The reinforcement 8 naturally must also comply with the standards in force concerning elements suitable for being implanted in the human body.

The reinforced fraction of the intragastric balloon 1 in accordance with the present disclosure presents a color that is different from that of the remainder of the balloon 1 so as to make it easier to locate the reinforced fraction visually using an endoscope.

This technical measure enables a surgeon in charge of extracting the balloon 1 to be certain to identify quickly the zones where the surface envelope 3 can and must be gripped with the help of an endoscopic extractor tool 2.

Preferably, this variation in color can be provided by the reinforcement 8 itself, which may, for example, present a dark color that can be seen by transparency through the surface envelope 3, which is conventionally pale in color, and preferably substantially translucent (when the envelope 3 is made of silicone).

More generally, the reinforcement 8 presents a color that differs from the color of the remainder of the balloon 1.

The present disclosure also provides a kit comprising both an intragastric balloon 1 in accordance with the present disclosure and an endoscopic extractor tool 2.

In other words, the kit comprises an intragastric balloon for treating obesity, that is to be implanted in the stomach of a patient in order to reduce the volume of the stomach, the balloon 1 being defined by a surface envelope 3 and an endoscopic extractor tool 2, with a fraction of the surface envelope 3 being associated with reinforcement 8 so as to form a reinforced fraction dedicated to extraction of the balloon 1 from the stomach by the reinforced fraction being gripped with the help of the endoscopic extractor tool 2.

The endoscopic extractor tool 2 includes a clamp, e.g., made of metal wires, as shown in FIG. 1.

Preferably, the clamp comprises two jaws 13, 14 connected to a wire guide 15, the wire guide sliding inside a catheter 16. In the absence of any mechanical stress, and as shown in the FIG. 1, the jaws 13, 14 tend to remain in a resilient return position in which they are spaced apart from each other, so the jaws of the clamp are open. When axial traction is exerted (along arrow 17) on the wire guide 15, while the catheter 16 is held stationary, then the jaws 13, 14 are forced into the catheter 16, thereby moving the jaws 13, 14 towards each other, and thus closing the jaws of the clamp.

Such an endoscopic clamp is well known to the person skilled in the art and it is not necessary to describe it in greater detail herein. Nevertheless, it should be observed that the endoscopic clamp used in the context of the present disclosure is preferably dimensioned so as to be suitable for being inserted in the operator channel of an endoscope having a section that is less than or equal to 2.8 millimeters.

Naturally, the kit may include an extractor tool other than a clamp without going beyond the scope of the present disclosure. In particular, known tools enabling a gripper function to be performed or a grasping function or a hooking function could be used.

The kit in accordance with the present disclosure operates as follows.

The balloon 1 is inserted in a folded configuration into the stomach of a patient endoscopically via the patient's mouth and esophagus.

The balloon is then inflated by blowing gas into the second pouch 5 via the valve 4. Inflation of the pouch 5 in turn inflates the first pouch 3B until the balloon 1 reaches its functional volume, which is substantially spherical in shape, for example.

The balloon 1 thus floats freely inside the patient's stomach, occupying the major fraction of the space usually available for food. The patient retains the balloon 1 within the stomach for a period that may cover several months. At the end of this period of treatment, the balloon 1 is deflated endoscopically, e.g., by being pierced. Thereafter, an endoscopic clamp 2 is inserted via the operator channel of the endoscope, heading towards the fraction 6, 7 of the surface envelope 3 that is provided with the reinforcement 8. Identifying such a reinforced fraction is made easier by the color of the reinforcement 8 being visible through the surface envelope 3, and also by the positioning of the reinforcement 8 close to the valve 4. The jaws 13, 14 of the clamp then grip the reinforced fraction of the surface wall 3, thereby mechanically engaging the jaws 13, 14 of the endoscopic clamp on the reinforcement 8.

This mechanical engagement enables the extractor tool to be secured firmly to the balloon, where such securing or hooking is sufficient to enable the balloon 1 to be pulled out from the patient's stomach without there being any risk of the balloon 1 escaping from the clamp as a result of tearing or the surface envelope 3 being damaged, as has been the case in the prior art.

The invention described in the present disclosure can be used in the fabrication and use of intragastric balloons for treating obesity.

1. An intragastric balloon for implanting in the stomach of a patient in order to reduce the volume of the stomach and thereby treat obesity, the balloon comprising a surface envelope wherein at least a fraction of the surface envelope is associated with a reinforcement for forming a reinforced fraction dedicated to extracting the balloon from the stomach by gripping the reinforced fraction with the help of an endoscopic extractor tool.

2. The intragastric balloon of claim 1, wherein the shape of the reinforcement matches substantially the shape of the fraction of the surface envelope with which it is associated.

3. The intragastric balloon claim 1, wherein the reinforcement is superposed on the fraction of the surface envelope with which it is associated.

4. The intragastric balloon of claim 3, wherein, for the fraction of the surface envelope presenting an inside face situated facing the inside of the balloon and an opposite outside face, the reinforcement is superposed on the inside face.

5. The intragastric balloon of claim 1, wherein the reinforcement is adhesively bonded to the fraction of the surface envelope with which it is associated.

6. The intragastric balloon of claim 1, wherein the reinforcement is disposed in the thickness of the fraction of the surface envelope with which it is associated.

7. The intragastric balloon of claim 1, wherein the reinforcement comprises at least one textile piece.

8. The intragastric balloon of claim 7, wherein the textile piece comprises a mesh that is suitable for encouraging the endoscopic extractor tool to hook onto the textile piece.

9. The intragastric balloon of claim 1, wherein the reinforcement comprises a film of plastics material.

10. The intragastric balloon of claim 1, wherein the balloon is expandable, and further comprises a valve associated with the surface envelope for connection to a source of fluid in order to expand the balloon inside the stomach by filling the balloon with the fluid, only a fraction of the surface envelope being associated with the reinforcement, the fraction comprising a first portion of the envelope situated in the vicinity of the valve.

11. The intragastric balloon of claim 1, wherein the reinforced fraction comprises a color that differs from the color of the remainder of the balloon, in order to make the balloon easier to locate visually by endoscopic means.

12. The intragastric balloon of claim 1, comprising at least first and second flexible pouches, the second flexible pouch being disposed inside the first pouch, the surface envelope forming the first pouch.

13. A kit, comprising:
a) an intragastric balloon for implanting in the stomach of a patient in order to reduce the volume of the stomach thereby treating obesity, the balloon comprising a surface envelope, and
b) an endoscopic extractor tool,
wherein a fraction of the surface envelope is associated with a reinforcement, for forming a reinforced fraction that is dedicated to extracting the balloon from the stomach by gripping the reinforced fraction with the help of the endoscopic extractor tool.

14. The kit of claim 13, wherein the endoscopic extractor tool comprises a clamp.

15. The intragastric balloon of claim 2, wherein the reinforcement is superposed on the fraction of the surface envelope with which it is associated.

16. The intragastric balloon of claim 9, wherein the film comprises a thermoplastic elastomer polyurethane.
